(19) United States
(12) Patent Application Publication
(10) Pub. No.: US 2005/0180982 A1
(43) Pub. Date: Aug. 18, 2005

(54) Cadherin-11 expression, an assay and treatment for cellular invasiveness

(75) Inventor: Colin D. MacCalman, Vancouver (CA)

Correspondence Address: MARSHALL, GERSTEIN & BORUN LLP, 233 S. WACKER DRIVE, SUITE 6300, SEARS TOWER, CHICAGO, IL, 60606, US

(73) Assignee: THE UNIVERSITY OF BRITISH COLUMBIA, Vancouver (CA)

(21) Appl. No.: 11/108,215

(22) Filed: Apr. 18, 2005

Related U.S. Application Data
(63) Continuation of application No. 09/830,811, filed on Aug. 30, 2001, now abandoned.
(60) Provisional application No. 60/106,258, filed on Oct. 30, 1998.

(51) Int. Cl. A61K 48/00; A61K 39/395; A61K 31/56

(52) U.S. Cl. 424/155.1; 514/44; 514/169

(57) ABSTRACT

A method of modulating differentiation or neoplastic transformation of cells is provided in which the cells are caused to increase or decrease cad-11 expression or function. Such method has application in affecting differentiation or neoplastic transformation of cells, preventing or terminating pregnancy by altering cad-11 function or expression in trophoblast cells, or for reducing the viability of carcinoma cells having a low to moderate metastatic potential. The use of agents which increase or decrease cad-11 expression or function is also provided, including such use for preparation of medicaments for modulating differentiation or neoplastic transformation of cells. A method for assessing the metastatic potential of carcinoma cells is also provided.

This invention relates to modulation of cadherin-11 expression in trophoblast cells and in carcinoma cells to affect differentiation or neoplastic transformation of cells. This invention also relates to assessment of the metastatic potential of epithelial rumors.

### BACKGROUND OF THE INVENTION

To establish a successful pregnancy, the trophoblast cells of the pre-embryonic blastocyst must interact with the uterine endometrium during a defined period of the menstrual cycle (called the window of implantation). Outside of this receptive period, the endometrium discourages implantation. Aberrant or “out of phase” development of the endometrium during the menstrual cycle has been associated with implantation failure, one of the factors believed to be underlying RSA and infertility. The adhesive mechanisms involved in establishing a uterine environment which promotes trophoblast-endometrial cell interactions have been poorly characterized.

The first step in implantation of the human pre-embryonic blastocyst involves the attachment of trophoblast cells of the blastocyst to surface epithelium of the uterine endometrium. Afterwards, the trophoblast cells proliferate and invade into the underlying endometrial stroma. The trophoblast cells differentiate into chorionic villi which are composed of two layers: the inner cell layer, which comprises mitotically active cytotrophoblasts, and the outer syncytial trophoblast, which is a terminally differentiated multi-nucleated cell formed by the fusion of post-mitotic cytotrophoblasts. As pregnancy proceeds, the cytotrophoblasts proliferate and form columns which extend through the syncytial trophoblast layer into the maternal decidua. These extravillous cytotrophoblast columns are believed to anchor the placenta to the decidua. Cytotrophoblasts dissociate from the extravillous columns and invade deeply into the maternal vasculature and decidua. These invasive cytotrophoblasts subsequently undergo differentiation and fusion to form placental bed giant cells, large multinucleated cells which lie in intimate contact with the surrounding decidual cells. During invasion of the endometrium, the trophoblast cells must not only interact with one another but with the diverse populations of cell types that constitute the endometrium.

The steroid hormones progesterone (P4) and 17-estradiol (E2) play a central role in preparing the endometrium for implantation. One of the steps involved in preparing the endometrium involves the differentiation of the stromal cells into decidual cells, which anchor the trophoblast cells and arrest their invasive migration. Morphologically, decidualization is characterized by a change of the stromal cells to a polyhedral cell shape with an increase in cell size. Ultrastructurally, there is extensive development of the organelles involved in protein synthesis (rough endoplasmic reticulum) and secretion (Golgi apparatus), and the appearance of desmosomes and gap junctions between adjacent cells.

The depth of trophoblast invasion is precisely controlled, and errors have extreme consequences to the health of the mother and fetus. For example, shallow invasion is associated with preeclampsia, a disease with significant maternal and fetal morbidity and mortality. In contrast, the absence of decidua allows trophoblasts to invade deeply into the underlying tissue as is the case in placenta accreta or ectopic pregnancy.

Alteration of cell-cell interaction is also a feature of neoplastic transformation of healthy cells to malignant rumor cells. Tumor metastasis requires first the disassociation of cells within the rumor, followed by invasion of the disassociated tumor cells into the underlying stroma followed by extravasion of the rumor cells into the blood stream or lymph. This is followed by re-association of the rumor cells at distant specific sites where secondary rumors are established. Thus, the process of neoplastic transformation has features in common with that of embryogenesis, including the differentiation of trophoblast cells as described above.

The cadherins are a gene superfamily of integral membrane glycoproteins that mediate calcium-dependent cell adhesion in a homophilic manner. The spatiotemporal expression of cadherin subtypes is highly regulated during development. Embryonic cells displaying different classical cadherins (type I cadherins) segregate from one another and it is believed that these cadherins provide the molecular basis for the segregation of discrete populations of cells and the subsequent formation of tissues. In the adult, the cadherins are localized to the membrane domains of the adherens junction and are believed to maintain the differentiated state of the cell.

Cadherins play a role in the alteration of cell-cell associations, both in embryogenesis and in rumor development. Cadherin expression has been linked to the invasive and metastatic potential of tumor cells. In generals lower levels of cadherins seem to be linked to a higher potential for rumor cells to disassociate and invade other tissue.

The classical cadherins include the E-, P-, and N-cadherins which mediate calcium dependent cell-cell adhesion in a variety of cell types. Aberrant expression of these classical cadherins have been associated with some rumors capable of spontaneous metastasis leading to the suggestion that there may be a link between heterogenous, expression of cadherins and metastatic potential (see: Takeichi, M. (1993) “Cadherins in Cancer: Implications for Invasion and Metastasis”. Current Opinion in Cell Biology 5:806-811).

E-cadherin (or epithelial cadherin) is involved in epithelial cell adhesion. Loss of E-cadherin has been associated with increased invasiveness and metastasis of epithelial derived rumors (carcinomas), including prostate (see: Umbas, R. et al. (1992) “Expression of the Cellular Adhesion Molecule E-cadherin is Reduced or Absent in High Grade Tumors”. Cancer Research 52:5104-5109; and breast (see: Oka. H. et al. (1993) “Expression of E-cadherin Cell Molecules in Human Breast Cancer Tissues and its Relationship to Metastasis”; Cancer Research 53:1696-1701.

Type 2 cadherins show low overall amino acid homology with classical cadherins. The type 2 cadherins share common sequence features, such as characteristic amino acid deletions or additions and distinctive amino acid substitutions at various sites, which are not found in classical cadherins. In particular, type 2 cadherins do not contain the cell adhesion recognition (CAR) sequence. HAV, which is conserved among all the classical cadherin subtypes. Cadherin-11 (cad-11), also known as OB-cadherin (OB-cad), is a type 2 cadherin which appears to play a central role in morphogenesis. [See U.S. Pat. No. 5,597,725 issued Jan. 28, 1997, incorporated herein; and, Takeichi, M. (1995) “Morphogenetic Roles of Classical Cadherins”, Curr. Opin. Cell. Biol. 7:619-627.]

Studies to date indicate that cad-11 expression is linked to differentiation of some cell types but this connection and any mechanisms whereby cad-11 mediates cellular differentiation is not understood.

It has been determined that cad-11 is expressed in the syncytial trophoblast but not the villous cytotrophoblasts of the human term placenta. Cad-11 expression is also detected in the cytotrophoblasts at the distal end of the extravillous cytotrophoblast column of the first trimester placenta. In the endometrium, cad-11 is spatiotemporally expressed in the glandular epithelium and stroma during the menstrual cycle. Levels of cad-11 in the glandular and surface epithelium remain relatively constant throughout the menstrual cycle. Cad-11 is not present in the stroma during the proliferative phase. Cad-11 is first detected around the spiral arteries of the stroma (the areas of early decidualization) during the late secretory phase. Cad-11 levels increase as the stroma continues to undergo decidualization and maximum levels are observed in the decidua of early pregnancy. [See MacCalman, et al. (1996) “Regulated Expression of Cadherin-11 in Human Epithelial Cells: A Role for Cadherin-11 in Trophoblast-Endometrium Interactions?”, Developmental Dynamics 206: 201-211; MacCalman et al., “Novel Cell Adhesion Molecules: Roles in Implantation?” from The Endometrium as a Target for Contraception. Beier et al., eds., Springer-Verlag. Berlin (1996), pages 137-157.]

The role of cad-11, if any, in the process of neoplastic transformation is not known. Cad-11 is thought to be a mesenchymal marker and its expression may be associated with progression of cancers that are characterized by a change from an epithelial to a mesenchymal phenotype. Cad-11 expression has been detected in signet-ring cell carcinomas exhibiting invasiveness and high peritoneal or stromal proliferation (see: Shibata. T. et al. (1996) “Simultaneous Expression of Cadherin-11 in Signet-Ring Cell Carcinoma and Stromal Cells of Diffuse-Type Gastric Cancer”: Cancer Letters (99:147-153). Also, in studies of different renal cell carcinoma cell lines. E-cad expression was associated with an epithelial phenotype whereas cad-11 expression was detected in cell lines having a fibroblastic phenotype, with no co-expression of E-cadherin and Cad-11 being detected in any single cell line (see: Shimazui. T. et al. (1996) “Complex Cadherin Expression in Renal Cell Carcinoma”: Cancer Res. 56:3234-3237).

It has now been found that increased cad-11 expression occurs during terminal differentiation and fusion of trophoblast cells whereby trophoblast cells become less invasive. Down regulation of cad-11 in such cells reduces the viability of syncytiallized trophoblast cells, and inhibits fusion and steroidogenic capacity of the cells. Thus, implantation or an established pregnancy may be disrupted by interfering with cad-11 expression.

It has also been determined that carcinoma cells which exhibit a limited metastatic potential express cad-11. Modulation of cad-11 expression in such cells will affect such cells capacity to either be invasive or attach to rumor sites. Furthermore, down regulation of cad-11 expression in such cells reduces their viability.

It has also been found that assessment of cad-11 as well as E-cadherin expression by carcinoma cells is an accurate indicator of the metastatic potential of such cells. Such an assessment based on cadherin measurement may replace tumor classification assessments based on only morphological grading of histological samples.

### SUMMARY OF THE INVENTION

This invention provides a method of modulating the differentiation or neoplastic transformation of 3 cell comprising the step of causing the cell to increase or to decrease cad-11 expression or function. Cad-11 expression may be increased by application of hormones such as progestin or TGF-1 which increase cad-11 expression. Cad-11 function may be decreased by contacting the cells with an agent that interferes with cad-11 function (eg. an anti-cad-11 antibody, preferably a neutralizing antibody or an antibody which inhibits either the cad-11 extracellular or intracellular domains when bound to cad-11). Cad-11 expression may be decreased by contacting the cells with an agent that interferes with cad-11 expression (eg. by preventing translation of cad-11 mRNA by use of a cad-11 antisense oligonucleotide or transcription by use of cad-11 sense or antisense oligonucleotides that will bind to the genomic DNA).

This invention also provides a method of preventing or terminating a pregnancy which comprises the step of decreasing cad-11 function or expression in human trophoblast cells. Cad-11 expression or function may be decreased as described above.

This invention also provides a method of reducing the viability of carcinoma cells having a low to moderate metastatic potential by decreasing cad-11 expression or function in the cells as described above. Cad-11 expression may be blocked by contacting the cells with a cad-11 antisense oligonucleotide whereby expression of cad-11 by the cells is interfered. The carcinoma cells may be prostate rumor cells.

This invention also provides a method for assessing the metastatic potential of carcinoma cells comprising the steps of contacting cells in a tissue sample suspected of containing a carcinoma with a detectable indicator capable of binding to cad-11 or cad-11 mRNA, and determining the presence or absence of cad-11 expression in the tissue. The method may further comprise the application of a second detectable indicator capable of binding to E-cadherin or E-cadherin in mRNA to cells of the tissue and determining the presence or absence of E-cadherin expression in the cells. The detectable indicators may be any substance which can be labelled for detection or which may be detected by its activity and Which has a specificity for binding the respective cadherin or mRNA, including an antibody and an oligonucleotide.

### DETAILED DESCRIPTION OF THE INVENTION

In this specification, the term “metastatic potential” means the relative propensity for the cells of a carcinoma to leave a tumor site and establish secondary “0 tumors. A “low to moderate” metastatic potential is characterized by cells exhibiting no propensity to invade the underlying stroma, up to a moderate level of such invasiveness. This state may be further characterized as existing in such cells which express one or the other of E-cadherin or cad-11, but not cells which have ceased expression of both cadherins.

Cells with a “high metastatic” potential have ceased expression of E-cad and cad-11. Such cells leave the tumor site via blood or lymph (extravasion) and will contribute to secondary tumor formations.

The term “carcinoma” means a rumor of epithelial origin, including an adenoma. These tumors include, but are not limited to carcinomas of the prostate, breast, lung, skin (eg. squamous cell carcinoma) as well as renal and gastric carcinomas.

The terms “cad-11 expression” and E-cadherin expression” mean the capacity or state of a cell whereby it produces the type 2 cadherin: cad-11, or the classical epithelial cadherin: E-cadherin. The terms “cad-11 function” and E-cadherin function” means the biological function of the respective cadherin.

The term “differentiation” means the process whereby there is a change in phenotype or function of a cell or in descendants of a cell, the term “neoplastic transformation” means the process whereby rumor cells become malignant or of an increased metastatic potential. Differentiation and neoplastic transformation may be characterized by a change in the degree of specialization of a cell, a change in the tendency of a cell to associate with other cells or a change in the mobility of a cell. The term “invasiveness” means the relative propensity of a cell to move into surrounding tissue.

Cad-11 and E-cadherin expression may be detected through use of a detectable indicator specific for the respective cadherin. The detectable indicator may be an antibody with specificity for binding to the targeted cadherin to which a detectable label or biological moiety may be attached. The label may be any substance that can be detected including a radio-label or dye. The label or biological moiety may be an enzyme or other active moiety that may be detected, for example by its capacity to emit light when stimulated. An antibody bound to the cadherin (first antibody) may also be detected by probing with a second antibody specific for the first antibody and the binding of the first and second antibodies is the detectable indicator. This may be facilitated by the detection of a label or biological moiety attached to the second antibody. Antibodies to E-cadherin and cad-11 are available and methods for producing such antibodies are known in the art (see: U.S. Pat. No. 5,597,725: Tanihara et al. [infra]: and Shimoyama. Y. et al. (1989) Cancer Res. 49:2118-2133).

A detectable indicator for E-cadherin or cad-11 may also be an oligonucleotide, preferably a short DNA sequence complementary to a unique portion of the m-RNA for E-cadherin or cad-11. The m-RNA sequences of E-cadherin and cad-11 are known, as are suitable complementary oligonucleotides, including oligonucleotides based in cDNA portions unique for either E-cadherin and cad-11 (see: Bussemakers. M. J. G. et al. (1993) “Molecular Biology Reports” 17:123-128; Tanihara, H. et al. (1994) Cell Ades. Commun. 2:15-26; and, U.S. Pat. No. 5,597,725). Methods for making such oligonucleotides, labelling the oligonucleotides and detecting such oligonucleotides when bound to a complementary mRNA sequence by base-pairing are known in the art.

Antisense oligonucleotides are short (eg. 7-50, preferably 13-0 bases) DNA or RNA molecules made with a sequence which is intended to be complementary to unique sequences in a gene or mRNA for a particular protein. The antisense sequence is intended to bind to its complementary sequence by base-pairing. Topically, the antisense is intended to bind to m-RNA thereby forming a heteroduplex which interferes with translation of the m-RNA or facilitates its cleavable by nucleases such as RNAseH. Methods for preparation of oligonucleotides comprising antisense intended to bind to genomic DNA to interfere with transcription are known, as are methods for preparation of catalytic ribozymes which selectively bind and cleave RNA molecules. Means for delivering such antisense constructs to cells are also known including encapsulation in liposomes, attachment of lipid moieties to the antisense or attachment of ligands for transmembrane receptors (such as transferrin and folate). Methods for determining the sequence of candidate oligonucleotides and their synthesis are known in the art. Candidate oligonucleotides are tested for their ability to bind to the target sequence with minimal interference (eg. by folding, loop formation, or proteins bound to the target sequence), and for the ability to interfere with expression of the target protein.

Antisense oligonucleotides of at least about 15 bases and complementary to unique regions of a DNA sequence encoding a cad-11 polypeptide can be synthesized by consensual technique. These include techniques for chemical synthesis such as, eg. by solid phase phosphoamite chemical synthesis. Alternatively, antisense RNA molecules can be generated by in vitro or in vivo transcription of DNA sequences encoding the RNA molecule. Such DNA sequence can be incorporated into a wide variety of vectors which incorporate suitable RNA polymerase promoters such as the T7 or SP6 polymerase promoters. Various modifications to the oligonucleotides of the present invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Delivery, of a antisense oligonucleotide to a particular tissue or organ may be accomplished, for example, by recombinant vectors and non-vector systems. Examples of non-vector systems suitable for delivery of antisense itself, include but are not limited to any lipid-based, lipid encapsulated DNA or cationic lipid/DNA complexes. Examples of recombinant viral vectors include but are not limited to herpes virus, retrovirus, vaccinia virus, adenovirus, and adenoassociated virus which are particularly suited for delivery of a gene encoding antisense. The recombinant vector delivery system will be typically formulated in a buffer comprising an agent which enhances delivery to a tissue or organ Examples of such delivery-enhancing agents are detergents, alcohols, glycols, surfactants, bile salts, heparin antagonists, cyclooxygenase inhibitors, hypertonic salt solutions, and acetates. Alcohols include for example, the aliphatic alcohols such as ethanol. N-propanol, isopropanol, butyl alcohol, acetyl alcohol. Glycols include glycerine, propyleneglycol and other low molecular weight glycols such as glycerol and thioglycerol. Acetates such as acetic is acid, gluconol acetate, and sodium acetate are further examples of delivery-enhancing agents. Hypertonic salt solutions like 1M NaCl are also examples of delivery-enhancing agents. Examples of surfactants are sodium dodecyl sulfate (SDS) and lysolecithin, polysorbate 80, nonylphenoxypolyoxyethylene, lysophosphatidylcholine, polyethylenglycol 400, polysorbate 80, polyosyethylene ethers, polyglycol ether surfactants and DMSO. Bile salts such as taurocholate, sodium taruo-deosycholate, deoxycholate, chenodesoxycholate, glycocholic acid, glycochenodeoxycholic acid and other astringents like silver nitrate may be used. Heparin-antagonists like quaternary amines such as prolamine sulfate may also be used. Cyclooxygenase inhibitors such as sodium salicylate, salicylic acid, and non-steroidal antiinflammatory drug (NSAIDS) like indomethacin, naproxen, diclofenac may be used.

The delivery-enhancing agent may be included in a buffer in which a recombinant adenoviral vector delivery system is formulated. The delivery-enhancing agent may be administered prior to the recombinant virus or concomitant with the virus. In some embodiments, the delivery-enhancing agent is provided with the virus by mixing a virus preparation with a delivery-enhancing agent formulation just prior to administration to the patient. In other embodiments, the delivery-enhancing agent and virus are provided in a single vial to the caregiver for administration.

In the case of a pharmaceutical composition comprising a gene contained in a recombinant adenoviral vector, the delivers system may be formulated in a buffer which further comprises a delivery-enhancing agent. The pharmaceutical composition may be administered over time, for example in the range of about 5 minutes to 3 hours. The delivery-enhancing agent may also be administered prior to administration of a recombinant adenoviral vector delivery system.

Concentration of the delivery-enhancing agent will depend on a number of factors known to one of ordinary skill in the art such as the particular delivery-enhancing agent being used, the buffer, pH, target tissue or organ and mode of administration. The concentration of the delivery-enhancing agent may be in the range of 1% to 50% (v/v). Preferably, any detergent in the final formulation administered to the patient is about 0.5-2× the critical micellization concentration (CMC).

The buffer containing the delivery-enhancing agent may be any pharmaceutical buffer such as phosphate buffered saline or sodium phosphate/sodium sulfate, Tris buffer, glycine buffer, sterile water and other known buffers. The pH of the buffer in the pharmaceutical composition comprising an adenoviral vector delivery system, may be in the range of 6.4 to 8.4.

For contraceptive applications, an agent that decreases cad-11 expression or function may be administered to a female subject by contacting the interior of the uterus with the agent. The agent may be an appropriate non-vector delivery system comprising cad-11 antisense.

A. Cad-11 Expression in the Differentiation of Endometrial Stromal Cells

The stromal cells of the human endometrium undergo terminal differentiation to form decidual cells during the luteal phase of the menstrual cycle. These differentiated endometrial cells play an important role in the establishment and maintenance of pregnancy. Aberrant development of the endometrium during the luteal phase is associated with implantation failure and placental dysfunction.

Decidualization is characterized by morphological and functional changes in endometrial stromal cells. Morphological dedidualization includes a chance in cell shape and size, extensive development of the organelles involved in protein synthesis, and secretion and the formation of desmosomes and gap junctions. Functionally, decidualization is characterized by the onset of prolactin (PRL) an insulin-like growth factor binding protein-1 (IGFBP-1) secretion. These differentiation processes have been shown to be regulated primarily by progesterone (P4) and 17-estradiol (E2). Cultured endometrial stromal cells undergo morphological and biochemical changes that are characteristic of decidualization when cultured in the presence of gonadal steroids.

Tissues

All of the tissues used in these studies were obtained with approval by the Committee for Ethical Review of Research involving Human Subject of the University of British Columbia.

Endometrial Stromal Cell Preparation and Culture

Endometrial tissue biopsies (n=6) were obtained from women of reproductive age during the mid-secretory phase of the menstrual cycle. All patients had normal menstrual cycles and had not received hormones for at least three months prior to the collection of tissue. The stage of the menstrual cycle was determined by the last menstrual period and confirmed by histological evaluation.

The endometrial stromal cells were separated from the glandular epithelium by enzymatic digestion and mechanical dissociation. The endometrial biopsy specimens were minced and subjected to 0.1% colagenase (type 1A. Sigma Chemical Co., St. Louis. MO) and 0.1% hyaluronidase (type I-S, Sigma Chemical Co.) digestion in a shaking water bath at 37° C. for 1 hour. The cell digest was then passed through a nylon sieve (38 m). The isolated glands were retained on the sieve and the eluate containing the stromal cells collected in a 50 ml tube. The stromal cells were pelleted by centrifugation at 800×g for 10 minutes at room temperature. The cell pellet was washed once in phenol red-free Dulbecco's Modified Eagle's medium (DMED) containing 10% charcoal-stripped fetal bovine serum (FBS) before being resuspended and plated in DMED containing 25 mM glucose, 25 mM Hepes, 1% (w/v) L-glutamine, antibiotics (100 U/ml penicillin, 100 g/ml streptomycin and 2.5 g/ml fungizone), and supplemented with 10% charcoal-stripped FBS. The culture medium was replaced 30 minutes after plating in order to reduce epithelial cell contamination. The purity of the cell cultures was determined by immunocytochemical staining for vimentin, cytokeratin, muscle actin, and factor VII. As defined by these criteria, the stromal cell cultures contained <1% endometrial epithelial or vascular cells.

Hormone Treatments

Stromal cells were grown to confluence, washed with PBS, and cultured in DMEM supplemented with 10%, charcoal-stripped FBS and containing either P4 (1 mM). E2 (30 nM), or vehicle (0.1% ethanol). The culture medium was replaced every 24 hours. The cells were harvested for either Northern or Western blot analysis after 0, 2, 4, 6, 8, 10, or 12 days of culture in the presence or absence of the steroids. The culture medium was chanced every day. Stromal cells were also cultured in the presence of P4 (1 mM) plus E2 (30 nM) for 0, 2, 4, 6, 8, 10, or 12 days before being harvested for Northern or Western blot analysis.

Northern Blot Analysis

Total RNA was prepared from the cultured stromal cells by the phenolchloroform method of Chomczynski and Sacchi (1987) Analytical Biochemistry 162: 156-159. The RNA species were resolved by electrophoresis in 1% agarose gels containing 3.7% formaldehyde. Approximately 20 g of total RNA was loaded per lane. The fractionated RNA species were then transferred onto charged nylon membranes.

The Northern blots were hybridized with radiolabelled cDNA probes specific for human cad-11 according to the methods of MacCalman et al. (1992) Developmental Dynamics 195:127-132. The blots were then washed twice with 2×SSPE at room temperature, twice with 2×SSPE containing 1% SDS at 55° C. and twice with 0.2×SSPE at room temperature. To standardize the amounts of total RNA in each lane, the blots were then probed with radiolabelled synthetic oligonucleotide specific for 18S rRNA according to the protocols described by MacCalman et al. (1992) [supra]. The blots were again subjected to radioautography to detect the hybridization of the radiolabelled probe to the 18S rRNA. The radioautograms were then scanned using and LKB laser densitometer. The absorbance values obtained for cad-11 mRNA transcripts were normalized relative to the 18S rRNA absorbance value.

Western Blot Analysis

For Western blot analysis, the stromal cells were washed with PBS and incubated in 100 l of cell lysis buffer (Tris-HCl, pH 7.5 containing 0.5% Nonidet P-40, 0.5 mM CaCl₂ and 10.0 mM PMSF) at 4° C. for 30 minutes on a rocking platform. The cell lysates were centrifuged at 10.000×g for 20 minutes and the supernatant used in the Western blot analysis. Aliquots (20 g) were then taken from the samples and subjected to SDS polyacrylamide eel electrophoresis under reducing conditions, as described by Laemmli (1970). The stacking gels contained 5% acrylamide and the separating gels were composed of 7.5% acrylamide. The protein were electrophoretically transferred from the gels onto nitrocellulose paper according to the procedures of Towbin et al. (1979) P.N.A.S. U.S.A. 76:43504354. The nitrocellulose blots were probed with a mouse monoclonal antibody (C11-113H) directed against human cad-11 (ICOS Corporation. Bothell. WA. ATCC accession number HB-12514). The Amersham ECL system was used to detect antibody bound to antigen. The radioautograms were then scanned using an LKB laser densitometer.

Statistical Analysis

The results are presented as the mean relative absorbance (+SE) for three independent experiments. Statistical differences between time points and treatments were assessed by the analysis of variance (ANOVA). Differences were considered to be significant for p<0.05. Significant differences between the means were determined using the least significant test.

A single cad-11 mRNA transcript of 4.4 kb was detected in all of the total RNA extracts prepared from the cultured endometrial stromal cells. P4 caused a significant increase in stromal cad-11 mRNA levels after 2 days of culture in the presence of this steroid. The levels of the cad-11 mRNA transcript continued to increase with time in culture with maximum cad-11 mRNA levels being observed in stromal cells cultured for 12 days in the presence P4. In contrast, E2, or vehicle alone (0.1% ethanol) did not significantly increase stromal cad-11 mRNA levels.

There was a significant increase in cad-11 mRNA levels after 2 days of culture in the presence of P4 plus E2. Maximum levels of cad-11 were observed after 10 days of culture in the presence of these two gonadal steroids. The levels of the cad-11 mRNA transcript remained elevated until the duration of these studies at 12 days of culture in the presence of these steroids. The increase in cad-11 mRNA levels in these cell cultures was greater than those observed in stromal cells cultured in the presence of P4 alone.

A single cad-11 protein species (Mr 125 kDa) was detected in all of the stromal cell extracts using the mouse monoclonal antibody directed against human cad-11 in Western blot analysis. In agreement with the Northern blot analysis, an increase in cad-11 expression occurred after 2 days of culture in the presence of P4. Maximum cad-11 expression levels were detected in extracts prepared from cell cultures in the presence of P4 for 12 days. E2 plus P4 caused a significant increase in cad-11 expression at 2 days of culture with maximum levels being observed after 10 days of culture in the presence of these steroids. No significant increase in cad-1 expression was detected in endometrial stromal cells cultured in the presence of E2, or vehicle alone. Thus, cad-11 mRNA and protein expression levels are tightly regulated during the steroid-mediated differentiation of cultured endometrial stromal cells.

B. Correlation of Cad-11 Expression with the Differentiation of Human Trophoblasts

Implantation is dependent on the proliferation, differentiation, and invasion of fetal trophoblast cells into the maternal endometrium. Growth factors and cytokines are believed to be key regulators of trophoblast differentiation and invasion, for example, the localization of transforming growth factor-(TGF-1) to the decidual cells of the human endometrium and the syncytial trophoblast and extravillous cytotrophoblast columns of the first trimester placenta has led to the proposal that this growth factor regulates the invasive capacity of trophoblasts in both an autocrine and paracrine manner. Furthermore, TGF-1 has been shown to reduce proliferation and promote the differentiation and fusion of highly invasive extravillous cytotrophoblasts into syncytium in vitro. These developmental processes have been associated with a reduction in the invasive capacity of these cells.

Cell Preparation and Culture

First trimester placentae were obtained from elective terminations of pregnancies (8-13 weeks). Extravillous cytotrophoblasts were propagated from first trimester placental explants as described by Graham et al. Biology of Reproduction 46:561-572. The chorionic villi were washed thoroughly in Dulbecco's Modified Eagle's Medium (DMEM; Gibco BRL. Burlington. Ont.) containing 25 mM glucose, 25 mM Hepes and 50 g/ml gentamicin. The villi were minced finely and plated in 25 cm² tissue culture flasks containing DMEM supplemented with antibiotics and 10% heat-inactivated fetal calf serum (FCS). The fragments of chorionic villi were allowed to adhere for 203 days, after which the non-adherent material was removed. The villous explants were cultured for a further 10-14 days, with the culture medium being replaced every 3 days. The propagated extravillous cytotrophoblasts were separated from the villous explants by a trypsin digestion (0.125% trypsin in Ca²⁻, Mg²⁻-free PBS) and plated in 60 mm culture dishes containing DMEM supplemented with antibiotics and 10% FCS. The purity of the extravillous cytotrophoblast cultures was determined by cytokeratin immunostaining. Only cultures which were 100% positive for cytokeratin immunostaining were used.

Extravillous cytotrophoblasts grown to 70% confluence in DMEM containing 10% FCS, were washed with PBS and cultured in serum-free DMEM for a further 24 hours. This culture medium was removed and after the cells had been washed twice with PBS, replaced with serum-free DMEM containing porcine TGF-1 (1 pg-10 ng/ml); Sigma Chemical Co., St. Louis. MO) or vehicle (40 M HCI/BSA). The cells were fixed for morphological assessment and immunocytochemistry or harvested for Northern or Western blot analysis after 24 hours of culture in the presence or absence of TGF-1.

Northern Blot Analysis

Total RNA was prepared from the cytotrophoblast cultures by the phenolchloroform method Chomczynski and Sachhi (1987) [supra]. The RNA species were resolved by electrophoresis in 1% agarose gels containing 3.7% formaldehyde. Approximately 20 g of total RNA was loaded per lane. The fractionated RNA species were then transferred onto charged nylon membrane (Amersham Canada Ltd., Oakville, Ont.).

The Northern blots were probed with a human cad-11 cDNA probe according to the methods of MacCalman et al. (1992) [supra]. The blots were then washed twice 2×SSPE (20×SSPE consists of 0.2 M sodium phosphate monobasic, pH 7.4 containing 25 mM EDTA and 3 Nt NaCl) at room temperature, twice with 2×SSPE containing 11% SDS at 55° C. and twice with 0.2×SSPE at room temperature. To standardize the amount of total RNA in each lane, the blots were then probed with a radiolabelled synthetic oligonucleotide specific for 18 rRNA according to the protocols described by MacCalman et al. (1992) [supra]. The blots were again subjected to radioautography to detect the hybridization of the radiolabelled probe to the 18S rRNA. The radioautograms were then scanned using an LKB laser densitometer. The absorbance values obtained for the cad-11 mRNA transcript were normalized relative to the 18 rRNA absorbance value.

Western Blot Analysis

For Western blot analysis, the trophoblasts were incubated in 100 l of cell lysis buffer (Tris-HCl, pH 7.5 containing 0.5% Nonidet P40, 0.5 mM CaCl₂ and 1.0 mM PMSF) at 4° C. for 30 minutes on a rocking platform. The cell lysates were centrifuges at 10.000×g for 20 minutes and the supernatant used in the Wester blot analyses. Aliquots were then taken from the samples and subjected to SDS-polyacrylamide gel electrophoresis under reducing conditions, as described by Laemmli (1970) Nature 227:680-685. The stacking gels contained 5% acrylamide and the separating eels were composed of 7.5% acrylamide. The proteins were electrophorectically transferred from the gels onto nitrocellulose paper according to the procedures of Towbin et al. (1979) [supra]. The nitrocellulose blots were probed with the mouse monoclonal antibody C 11-113H described in MacCalman et al. 1996 [supra] and Getsios et al. 1998 Developmental Dynamics 211:238-247. The Amersham ECL system was used to detect antibody bound to antigen. The radioautograms were then scanned using an LKB laser densitometer.

Immunocytochemistry

The cells were washed twice in PBS and fixed in 0.1% glutaraldehyde/2% formaldehyde at 4° C. for 20 minutes Immunocytochemistry was performed using a mouse monoclonal antibody directed against human cytokeratin filaments 8 and 18 (Becton Dickson. San lose. CA) or a further monoclonal antibody directed against human cad-11 (C11-113E; ICOS Corporation. Bothell, Wash.; ATCC accession no. HB-12515) as described in (MacCalman et al. 1996 [supra] and Getsios et al. 1998 [supra]). Sequential incubations were performed according to the methods of Cartun and Pedersen (1989) J. of Histotechnology 12:273-280 and included 10% normal horse serum for 30 minutes, primary antiserum at 37° C. for 1 hours, secondary biotinylated antibody at 37° C. for 45 minutes, streptavidin-biotinylated horseradish peroxidase complex reagent at 37° C. for 30 minutes, and three five minutes washes in PBS. The cells were then exposed to chromogen reaction solution (0.035% diaminobenzidine and 0.03% H₂HO₂) for 10 minutes, washed in tap water for 5 minutes, counterstained in haematoxylin, dehydrated, cleared and mounted.

Statistical Analysis

The results are presented as the mean relative absorbance (±SE) for three independent experiments. Statistical differences between treatments were assessed by the analysis of variance (ANOVA). Differences were considered to be significant for p<0.05. Significant differences between the means were determined using the least significant test.

A single cad-11 mRNA transcript of 4.4 kb was detected in all of the total RNa extracts prepared from the cultured extravillous cytotrophoblasts. TGF-1 increased cad-11 mRNA levels in the cultured extravillous cytotrophoblasts in a dose-dependent manner. However, a significant increase in cad-11 mRNA levels was only observed in extracts prepared from trophoblasts cultured in the presence of 1 or 10 ng/ml of TGF-1. Maximum cad-11 mRNA levels were observed in extravillous cytotrophoblasts cultured in the presence of 10 ng/ml TGF-1.

Western blot analysis, using extracts prepared from extravillous cytotrophoblasts cultured in the presence or absence of TGF-1 and a mouse monoclonal antibody directed against human cad-11, revealed a single cad-11 protein species (Mr 125 kDa) in all of the cellular extracts. In agreement with the Northern blot analysis, TGF-1 caused an increase in cad-11 expression levels in a dose-dependent manner with a significant increase in cad-11 protein expression levels only being observed in cytotrophoblasts cultured in the presence of 1 or 10 ng/ml of TGF-1. Maximum cad-11 expression levels were observed in extracts prepared from cells cultured in the presence of 10 ng/ml TGF-1.

Extravillous cytotrophoblasts cultured in the presence or absence of TGF-1 for 24 hours were immunostained for cad-11. There was marked increase in the number of cells expressing cad-11 in cultures treated with 1 or 10 ng/ml of TGF-1 but not in cultures treated with lower concentrations of TGF-1. Cad-11 was further localized to the large cellular aggregates and multinucleated cells which were observed primarily in cultures treated with 1 or 10 ng/ml or TGF-1. These cellular structures were predominant in cultures treated with 10 ng/ml TGF-1.

Cad-11 expression was examined in cytotrophoblast cells isolated from term and first trimester placenta, and in three choriocarcinoma cell lines, BeWo, JEG-3, and JAR. Cytotrophoblast cells isolated from term placenta will readily undergo fusion to form syncytium in culture. Low levels of cad-11 were detected in freshly isolated cytotrophoblasts. However, as the mononucleate cytotrophoblasts aggregated and fused to form syncytium, there was a marked increase in cad-11 RNA levels. The increase in cad-11 mRNA levels was concomitant with a decrease in E-cadherin mRNA levels. Thus. E-cadherin is down-regulated ad the mononucleate cytotrophoblasts differentiate and fuse to form syncytium. In contrast, cad-11 levels increase as the trophoblast cells undergo terminal differentiation to form syncytial trophoblasts.

Treatment with TGF-1 increases cellular differentiation and reduce the invasive capacity of isolated extravillous cytotrophoblasts. The effect of porcine TGF-1 (10 pg-10 ng/ml. Sigma Chemical Co. MO) on cad-11 expression in these primary cell cultures was examined by Western blot analysis. TGF-1 increased cad-11 levels in a dose-dependent mariner in extravillous cytotrophoblasts. Large cellular aggregates, most of which contained multinucleated cells, were observed in cultures treated with 0.1 ng/ml of TGF-1 for 12 hours. The number and size of the cellular aggregates increased with continued culture in the presence of TGF-1. Intense immunostaining for cad-11 was detected in all of the cell aggregates which formed in the presence of high concentrations of TGF-1.

Thus, Cad-11 expression is highly regulated during trophoblast differentiation. In particular, cad-11 expression is associated with the fusion of villous mononucleate cytotrophoblasts isolated from term placenta, and with the aggregation and fusion of extravillous cytotrophoblasts in response to TGF-1 These differentiation processes are associated with a reduction in trophoblast invasiveness. Formation and organization of the extravillous cytotrophoblast column may be affected as well as trophoblast-decidual cell interactions whereby the placenta is anchored to the maternal decidua. Loss of cad-11 expression allows cytotrophoblasts to detach from the column and invade into the underlying maternal tissues.

Co-cultures of decidua parietalis and villous placenta were established on a three-dimensional gel of rat tail collagen. The decidua and the villous tissue were cut into 1-2 mm cubes, explants were placed in 35 nun culture dishes containing 0.1 ml of the collagen mixture. After the gel had formed, a piece of villous tissue, suspended in 0.05 ml of culture medium was placed on to the exposed decidual surface. After 2 hours, the plates were flooded with culture medium. Stable adhesion between the villous and decidual tissues occurred within 48 hours. Samples are harvested after 48, 72, or 96 hours of culture. Tissues were embedded in OCT compound, snap frozen, and frozen section prepared. Blocks containing the co-cultures were cut in-full in order to obtain information about the three dimensional aspect of the contact sites. Contact sites were identified by hematoxylin staining and confirmed by immunostaining for cytokeratin. In the areas of contact, there was cytotrophoblast layering leading to localized erosion of the syncytial trophoblast and the formation of an extravillous column, which penetrates the underlying decidual explant.

Cad-11 was expressed in decidua and placental explants. In particular, cad-11 expression was localized to the synctial trophoblast of all the villous tissue and in the extravillous cytotrophoblast columns which form in the areas of contact between the villous and decidual explants. The spatiotemporal expression of cad-11 in the cultured villous explants was consistent with that observed in first trimester placenta.

Antisense oligonucleotide sequences (18 mers, 50% AT/GC) were selected from the full-length cad-11 cDNA sequence using the MacVector™ program. DNA sequences located near the 5′ end of the cad-11 cDNA were selected and compared to the human sequence databases of GenBank and EMBL bank. Two sequences, which showed low homology (41.5%) with other known human DNA sequences were identified. OB-1, and OB-2 as shown in Table 1. Sense oligonucleotides, OB-3 and OB-4, were prepared as controls. Phosphothiorate labelled oligonucleotides were prepared at the Biotechnology Lab. of The University of British Columbia. The same results were obtained using OB-1 and OB-3, as with OB-2 and OB-4.

TABLE 1

| | | |
|---|---|---|
| OB-1: | GGC GGC TTG TAA ACA GTA | (SEQ ID NO:1) |
| OB-2: | CAC GAA GAA CTG GTT CCA | (SEQ ID NO:2) |
| OB-3: | ATG ACA AAT GTT CGG CGG | (SEQ ID NO:3) |
| OB-4: | ACC TTG GTC AAG AAG CAC | (SEQ ID NO:4) |

In order to determine the specificity of OB-1, the effect of OB-1 was determined in human granulosa cells. Human granulosa cells express three cadherin subtypes (cad-11, cad-6 and N-cad) as they undergo spontaneous luteinization in culture. Granulosa cells which had been cultured for 36 hours were treated with OB-1 or OB-3 (0.5-5 M). The cultures were maintained for a further 24 hours, after which the cells were harvested for Northern analysis. Northern blots were probed with cad-11, cad-6, or N-cadherin (neural cadherin) cDNA probes as previously described. OB-1 caused a reduction in the levels of the cad-11 RNA transcript in a dose-dependent manner but had no effect on cad-6 or N-cadherin mRNA levels. In contrast. OB-3 did not effect the levels of any of the three cadherin subtypes. Thus, OB-1 is specific for cad-11 and does not significantly effect mRNA levels of other type 2 cadherins (cad-6) or classical cadherins (N-cadherin).

Non-specific effects of the antisense oligonucleotides on cell viability and the steroidogenic capacity of cells was determined using Cos-1 cells transfected with the steroid acute regulatory protein (from Dr. J. F. Strauss, III. University of Pennsylvania, Philadelphia, Pa.). These cells, which do not express cad-11, are capable of producing high progesterone from cholesterol precursors. The COS-1 cells were cultured in the presence or absence of varying concentrations of OB-1 OR OB-3 (0.5-5 M) for 48 hours. The cell number present in each culture was determined using a Coulter™ cell counter and P4 concentrations present in the culture medium using a Progesterone RIA™ kit. As determined by these parameters. OB-1 or OB-3 did not have a significant effect on cell number of the steroidogenic capacity of these cells at any of oligonucleotide concentrations examined.

Mononucleate cytotrophoblasts isolated from human term placenta will aggregate and fuse to form syncytium with time culture. Cellular aggregation in these cultures is observed after approximately 12 hours, with syncytial structures being observed after approximately 24 hours. After 72 hours, multinucleated syncytium is the predominant cell feature in these cultures. As described above, the terminal differentiation and fusion of these cells is associated with an increase in cad-11 mRNA and protein expression levels and a concomitant decrease in E-cad expression. In addition, these developmental processes result in a marked increase in P4 and human chorionic gonadotropin (hCG) production.

The effects of OB-1 on the differentiation of these trophoblasts isolated from term placenta was determined. Cytotrophoblasts were cultured for 0, 6, 12 or 24 hours before being treated with either OB-1 or OB-3 (5 NI). The cultures were maintained for a further 24 or 48 hours, after which the cells were harvested for Western blot analysis. Trophoblasts cultured in the presence of OB-1 failed to up-regulate cad-11 with time in culture whereas E-cad levels remained constant throughout the duration.

The effect of OB-1 and OB-3 on the morphology of the cytotrophoblast was examined by light microscopy and indirect immunofluoresence. Large cellular aggregates were observed in all of the cell cultures suggesting that OB-1 did not appear to affect the ability of the cytotrophoblasts to aggregate. Indirect immunofluoresence using an monoclonal antibody directed against E-cadherin (Transduction laboratories, Lexington. KY) demonstrated that these cell aggregates were comprised of mononucleate cytotrophoblasts. The morphology of the cells present in the cellular aggregates suggested that the cells had undergone alignment, a phenomenon that precedes cellular fusion. In contrast, E-cadherin was not detected in the multinucleated syncytial structures which formed in the cultures treated with OB-3.

To determine whether reduced cad-11 expression in villous cytotrophoblasts would affect the functional differentiation of the primary cell cultures, concentrations of P4 and hCG were measured in the culture medium. The number of cells present in these cell cultures was determined by DNA content. The levels of hCG and P4 in the culture medium obtained from cells treated with OB-1 were significantly lower than those observed in medium obtained from OB-3 cells.

To determine whether the effects of OB-1 on villous cytotrophoblasts are reversible, cells were cultured in the presence of OB-1 or OB-3 for 48 hours. The cells were washed thoroughly with PBS. The culture medium was then replaced and the cells cultured for a further 24 hours. At the duration of these studies, large syncytial structures were observed in the OB-3 treated cultures. In contrast, the cells treated smith OB-1 were no longer viable. The majority of cells (90%) detached from the culture and dish. Cad-11 was not detected in the remaining cells.

Villous cytotrophoblasts isolated from the human term placenta were cultured for 72 hours before OB-1 or OB-3 were added to the culture medium. The cells were cultured for another 24 or 48 hours. In the duration of these studies, cells were stained with trypan blue, or fixed and examined using phase contrast microscopy. Large syncytial structures were observed in the cultures treated with OB-3. These syncytial structures were viable as determined by trypan blue staining. In contrast, cellular debris was observed in cultures treated with OB-1 for 24 hours. The syncytium was heavily fragmented and the syncytial structures were no longer viable. Large areas of cellular debris were fixed onto the culture dish, particularly in areas where the syncytial membrane appeared to have been completely lost.

Similar observations were observed in BeWo choriocarcinoma cells cultured in the presence of cAMP to induce cellular differentiation and fusion. Depending on the time of culture in the presence of cAMP, OB-1 was capable of inhibiting cellular differentiation and fusion in cells cultured in the presence of cAMP for 12 hours or disrupted the membrane integrity of the syncytial structures which formed after 24 hours.

In order to examine the effects of OB-1 and OB-3 on human decidual cells, primary cell cultures were treated with varying doses (0.5-5 M) of OB-1 or OB-3 for 24 hours. OB-3 did not have a significant effect on cell viability and number at any of the oligonucleotide concentrations used in this study. In contrast, OB-1 caused the cells to lose interactions with one another and detach from the plastic in a dose-dependent manner. Trypan blue staining of these cells demonstrated that these cells were no longer viable.

In order to determine whether the cad-11 antisense oligonucleotides could decrease cad-11 expression in tissue explants, decidual explants were cultured in the presence of OB-1 or OB-3 (5 M) for 24 hours. Western blot analysis demonstrated that cad-11 expression was significantly reduced in the decidual explants cultures in the presence of OB-1.

Thus, cad-11 expression in trophoblast cells is associated with terminal differentiation and loss of the invasive phenotype. Increased cad-11 expression in the highly invasive trophoblasts reduces the invasive capacity of these cells and promotes cellular aggregation and fusion. Cad-11 expression increases in extravillous cytotrophoblasts cultured in the presence of TGF-1. Failure to increase cad-11 expression during the terminal differentiation of villous cytotrophoblasts and BeWO cells cultured in the presence of cAMP will inhibit fusion. P4 and hCG production does not increase in trophoblasts cultured in the presence of the cad-11 antisense oligonucleotide. Prolonged culture of these cells in the presence of the oligonucleotide results in cell death.

Cad-11 expression is highly regulated during cellular differentiation and disruption in the expression pattern has a dramatic effect on the developmental fate and viability of cells. Inhibition of cad-11 expression may be used to disrupt the integrity and function of the placenta and decidua and to prevent pregnancy.

C. Regulated Expression of Type 2 Cadherins During Neoplastic Transformation of Prostate Cancer Cells

To associate cad-11 with prostate cancer cell progression in vivo, the expression of cad-11 was determined in prostate carcinomas with proven clinical outcomes. Routinely fixed and processed paraffin embedded tissue were obtained from 8 cases of radical prostatectomy for carcinoma. The tumors were diagnosed and assigned Gleason histopathological grades and scores using H&E stained sections. Gleason scoring is described below. Prostate glands were systematically sectioned and the sections mapped to facilitate the anatomical location and measurements of the rumors and their relation to the different areas of the prostate. The sections were immunostained for E-cadherin, and cad-11. It was found that E-cadherin and cad-11 are differentially expressed during the neoplastic progression of prostate cancer. E-cadherin expressed in cells of a low metastatic potential. Cad-11 was expressed in carcinoma specimens assigned an intermediate Gleason score, an assignment associated with the most difficult and unreliable prognosis. These cells have a moderate metastatic potential. Cad-11 was not detected in poorly differentiated carcinomas (high Gleason scores) which exhibit a high metastatic potential. In contract, cad-11 was readily detectable in a secondary bone tumor.

Similar to the invasive extravillous cytotrophoblasts N-cadherin, and cad-6 were found to be the predominant cadherin subtypes present in the highly aggressive TSU-Prl prostate cancer cell line (from K. Jarvi, Dept. of Urology, University of Toronto) and the PC3 prostate cancer cell line (from American Type Culture Collection) using RT-PCR. However, low levels of the mRNA transcripts encoding E-cadherin and cad-11 were also detected in these cell lines.

As TGF-1 has been shown to regulate cellular proliferation and invasion in prostate cancer cells, cad-11 expression in the cell lines cultured in the presence of TGF-1 (10 pg-1 ng/ml) for 24 hours was determined. Cad-11 mRNA levels were readily detectable in PC3 cells cultured in the presence of TGF-1 using semiquantiative PCR. The increase in cad-11 was concomitant with a decrease in cad-6 and N-cadherin mRNA levels.

The ability of the cad-11 antisense oligonucleotides to modulate cad-11 expression in PC3 cells treated with TGF-1 for 24 hours was determined. The cells were cultured in the presence of varying doses (0.5-5 M) of OB-1 or OB-3 for 24 hours. OB-3 did not have a significant effect on cell viability and number at any of the oligonucleotide concentrations used in this study. In contrast, OB-1 caused the cells to lose interactions with one another, and detach from the plastic in a dose-dependent manner. Trypan blue staining of these cells demonstrated that these cells were no longer viable.

Thus, cad-11 expression is tightly regulated during the neoplastic transformation of the carcinoma. E-cadherin and cad-11 are differentially expressed during the neoplastic transformation of these cells. In addition, cad-11 expression is restricted to carcinoma cells with low to moderate metastatic potential and is lost with continued dedifferentiation along the invasive pathway. Cad-11 is associated with attachment of the carcinoma to bone cells during the formation of secondary tumors on bone. The correlation of cad-11 with the reduced invasive capacity of these carcinoma cells in the primary or secondary tumor reflects the expression pattern observed in trophoblast cells. Similarly, cad-11 expression increases in PC3 cells cultured in the presence of TGF-1. This growth factor has been shown to reduce cellular proliferation and the invasive capacity of these cells. Antisense oligonucleotides reduce the viability of these cancer cells when the cells are at a stage of neoplastic transformation before cad-11 expression ceases.

D. Assessment of Carcinoma Metastatic Potential

The Gleason classification scheme which is routinely used to assess metastatic potential of prostate carcinoma, employs low-power magnification (X40-100) to assess the glandular pattern of the tumor and its relationship to the stromal compartment (eg. see: Tannenbaum. M. (ed); “Urologic Pathology: The Prostate”: Lee & Folger, Phil. Penn. USA, at p. 171-197). Five tumor grades progressing from the most (1) to the least (5) differentiated are recognized, the tumor grade(s) noted are recorded. A Gleason score consisting of the sum of the most and the next most prevalent grade is calculated.

Prostate samples obtained as described in the previous example were subjected to the Gleason classification system and monitored for cad-11 and E-cadherin expression according to the methods of that example.

Grade 1 pattern cells contain a rather homogeneous array of single, round to oval, separate but closely packed glands. Stromal invasion for such cells is infrequent, and when present, is expansile in nature. Tumor margins are very well defined. In these cells. E-cadherin was localized to the membrane of the glandular cells. Cad-11 was not detected.

Grade 2 pattern has an acinar pattern quite similar to that of pattern 1 except that there is less uniformity in glandular shape and often up to one diameter distance between acinar units. A mild amount of stromal invasion is encountered; consequently, the tumor margins are less well circumscribed. This stage, which is characterized by loss of glandular structure and shape, was associated with irregular E-cadherin expression (granular cytoplasmic immunostaining and not at the interface between glandular cells) or loss of E-cadherin expression. Cad-11 was readily detectable in the glandular structures which had lost E-cadherin expression. Slight cad-11 immunostaining was observed in some cells with irregular E-cad expression. In addition, cad-11 expression was detected in cells which had invaded into the stroma.

Grade 3 pattern contains three distinctive subpatterns. The first is characterized by single but very irregular glands separated from one another by more than one gland diameter. Stromal invasion is exhibited to a moderate degree, and the tumor margins are generally poorly defined. The second consists of microglandular nests of cells that form small groups. The third subpattern manifests sharply as circumscribed rounded masses of papillary epithelium possessing smooth sharp edges. They are seen as expansile masses within the stroma, are found in medium to large glands and are also associated with poorly defined tumor margins. Low levels of E-cad were detected in some of the irregular glandular structures. In contrast, cad-11 was readily detectable in all the three subpatterns of Gleason Grade 3.

Grade 4 pattern contains two subpatterns. The first consists of fusion of ragged glandular masses that exhibit prominent branching. Stromal invasion is marked and the tumor appears quite ill-defined. The second subpattern consists of the same morphologic type of rumor except for the presence of large cells, with very paly “hypernephroid” cells. Cad-11 was the primary cadherin subtype detected in Grade 4 patterns. In particular, cad-11 was detected in cells exhibiting the first histologic subpattern of Grade 4. In contrast, cad-11 was not detected in the “hypernephroid” cells.

Grade 5 pattern is characterized by irregular infiltrating masses of malignant cells, usually without any gland formation. This pattern is typified by extensive stromal invasion, a virtually imperceptible interface between the tumor and adjacent parenchyma. Negligible E-cad and cad-11 immunostaining was observed in Grade 5 rumors.

Approximately 50% of rumors will manifest more than one of Gleason's histologic pattern. This factor together with occurrence of histologically heterogeneous rumors led to the use of sums of the primary and secondary patterns as the Gleason score. Possible pattern scores range from 2 to 10 with the former being very indicative of very well-differentiated tumors and the latter of the highly is undifferentiated variety.

In a sample of tissues (n=18) assigned Gleason scores, it was determined that:

1. E-cadherin is the predominant cadherin subtype expressed in rumors assigned scores of 2-4 which have low metastatic potential.

2. Cad-11 is the predominant cadherin subtype expressed in tumors assigned scores 5-6(7) which have moderate metastatic potential.

3. Both cadherin subtypes are lost in Gleason scores (7) 8 or above, which have high metastatic potential.

Cad-11 expression in the Gleason (n=4) scored tumors with moderate metastatic potential was a predictor of subsequent tumor progression and metastasis. The Gleason scores themselves cannot predict the progression of the disease. Loss of cad-11 expression is an indicator of metastasis whereas expression, predicts containment of the tumor. Cells which still express E-cadherin or which have switched to cad-11 expression may be treated according to the methods of this invention by increasing cad-11 expression to affect their invasiveness or to decrease cad-11 expression or function to reduce their viability. Cells that express E-cadherin may be treated to decrease the potential for cad-11 expression and thus to decrease their viability when they progress to the stage of cad-11 expression. Cells that express cad-11 may be killed by treatment to decrease cad-11 expression or function. Alternatively, such cells may be treated to increase cad-11. Highly metastatic cells which no longer express cad-11 may not be killed in this fashion but may be stimulated to recommence cad-11 expression whereby the invasiveness of the cells will be reduced (eg. by treatment with factors such as TGF-1).

All publications, patents and patent applications referred to herein are hereby incorporated by reference. While this invention has been described according to particular embodiments and by reference to certain examples, it will be apparent to those of skill in the art that variations and modifications of the invention as described herein fall within the spirit and scope of the attached claims.

1. A method of modulating differentiation or neoplastic transformation of cells by causing said cells to increase or decrease cad-11 expression or function.

2. The method of claim 1 wherein said modulation is of differentiation or neoplastic transformation of the cell.

3. The method of claim 1 wherein said modulation is for preventing or terminating a pregnancy, and said method comprises decreasing cad-11 function or expression in trophoblast cells.

4. The method of claim 1 wherein said modulation is for reducing the viability of carcinoma cells having a low to moderate metastatic potential, and said method comprises decreasing cad-11 expression or function in the cells.

5. The method of any one of claims 1-4 wherein cad-11 expression is increased by hormone treatment of the cells.

6. The method of any one of claims 1-4 wherein cad-11 function is decreased by contacting the cells with an agent that interferes with cad-11 function.

7. The method of claim 6 wherein said agent is an anti-cad-11 antibody.

8. The method of any one of claims 1-4 wherein cad-11 expression is decreased by contacting the cells with an agent that interferes with cad-11 expression.

9. The method of claim 8 wherein said agent prevents transcription to or translation of, cad-11 mRNA.

10. The method of claim 9 wherein said agent is an antisense oligonucleotide.

11. The method of claim 10 wherein said oligonucleotide comprises an oligonucleotide substantially identical to, or which will hybridize to SEQ ID NO:1.

12. The method of claim 4 wherein said carcinoma cells are prostate rumor cells.

13. The use of a hormone for preparation of a medicament for use in the method of any one of claims 1-12

14. The use of an agent that interferes with cad-11 function for preparation of a medicament for use in the method any one of claims 1-12.

15. The use according to claim 14 wherein said agent is an anti-cad-11 antibody.

16. The use of an agent that interferes with cad-11 expression for preparation of a medicament for use in the method of any one of claims 1-12.

17. The use of claim 16 wherein said agent is an antisense oligonucleotide which prevents transcription to, or translation of cad-11 mRNA.

18. The use of claim 17 wherein said oligonucleotide comprises an oligonucleotide substantially identical to, or which will hybridize to SEQ ID NO:1.

19. A method for assessing the metastatic potential of carcinoma cells comprising: contacting cells in a tissue sample suspected of containing a carcinoma with a detectable indicator capable of binding to cad-11 or cad-11 mRNA, and, determining the presence or absence of cad-11 expression in the tissue.

20. The method of claim 19 wherein said detectable indicator comprises an oligonucleotide.


SEQUENCE LISTING (4 sequences)

SEQ ID NO: 1 - DNA, 18 bp, Homo sapiens
ggcggcttgtaaacagta

SEQ ID NO: 2 - DNA, 18 bp, Homo sapiens
cacgaagaactggttcca

SEQ ID NO: 3 - DNA, 18 bp, Artificial Sequence
  Description of Artificial Sequence Sense oligonucleotide
atgacaaatgttcggcgg

SEQ ID NO: 4 - DNA, 18 bp, Artificial Sequence
  Description of Artificial Sequence Sense oligonucleotide
accttggtcaagaagcac